(19) United States
(12) Patent Application Publication
(10) Pub. No.: US 2026/0176241 A1
(43) Pub. Date: Jun. 25, 2026

(54) PROCESS FOR THE PRODUCTION OF A CO-MIXTURE OF PIPERAZINE AND METHYL-SUBSTITUTED PIPERAZINE

(71) Applicant: UOP LLC, Des Plaines (IL)

(72) Inventors: Nabil Al Yassir, Ontario (CA); Carl J. Stevens, Seaside, CA (US); Edwin P. Boldingh, Arlington Heights (IL)

(21) Appl. No.: 19/397,137

(22) Filed: Nov. 21, 2025

Related U.S. Application Data
(60) Provisional application No. 63/737,390, filed on Dec. 20, 2024.

(51) Int. Cl. C07D 295/023; B01J 29/40; C07D 241/04; C07D 295/027

(52) U.S. Cl. CPC C07D 295/023; B01J 29/40; C07D 241/04; C07D 295/027

(57) ABSTRACT

Catalytic processes to manufacture piperazine and 2-methyl piperazine simultaneously through co-feeding a linear alkanolamine, or a diol, or an ethyleneamine with an analog of the alkanolamine, or the diol, or the ethyleneamine in the presence of anhydrous ammonia to produce a reaction mixture comprising the piperazine and the methyl-substituted piperazine are described. The catalyst comprises a 10 membered ring zeolite.

### RELATED APPLICATIONS

This application claims priority to U.S. Provisional patent application Ser. No. 63/737,390, filed on Dec. 20, 2024, the entirety of which is incorporated herein by reference.

### BACKGROUND

Cyclic diamine piperazine is one of the leading solvent candidates for CO₂ capture processes from post-combustion sources, such as flue gas, offered by multiple technology providers. Many other solvents are not suitable because they have lower CO₂ capacity and high levels of thermal and oxidative degradation that prevent operation at high temperatures.

Currently, piperazine is only made as a by-product of ethylene amines commercial technologies (viz. reductive amination of ethylene dichloride (EDC) or monoethanolamine (MEA)). As shown in Examples 1-3 and Tables 2-4, the combined amount of piperazine and aminoethylethanolamine is 75 wt % or greater of the products in the product composition. The piperazine is 10-40 wt % of the products in the product composition.

US Application No. 2014/0371452 describes a method for the reductive amination of diethanolamine to form a product composition that includes piperazine and aminoethylethanolamine along with other by-products. A catalyst with a transitional alumina/second metal oxide support and a mixture of catalytic metal is used for the reaction. As shown in Examples 1-3 and Tables 2-4, the combined amount of piperazine and aminoethylethanolamine in the product composition is 75 wt % or greater of the products. The piperazine in the product composition is 10-40 wt % of the products.

The market growth of ethylene amines is about 3.7% compound annual growth rate (CAGR) whereas piperazine growth is about 10-20% CAGR. It is expected that piperazine capacity will be insufficient to meet the demand if the market adopts piperazine for carbon capture processes.

In addition, piperazine has a low solubility in water which can prove detrimental in a large-scale system because of the increase in energy consumption. At ambient conditions, solid piperazine has a solubility less than 2 M in water, so it cannot be used in traditional systems at concentrations that give adequate CO₂ capacity for good energy performance. The aqueous piperazine solution for the targeted CO₂ absorption capacity for an advanced solvent carbon capture (ASCC) process was determined to be 30 wt. % (corresponding to 5 molal (m)). This necessitates operating at a temperature greater than or equal to 40° C. above the piperazine crystallization temperature which raises concerns with operational flexibility (i.e., upset conditions). Alternatively, maintaining the CO₂ loading for the fresh (lean) solution (mol CO₂/mol piperazine=0.23) implies a 25% loss in piperazine capacity at the starting point because the lean solvent contains 25% CO₂ to prevent precipitation of the piperazine.

Therefore, there is a need for a piperazine based solvent or mixture having increased solubility in water, increased yield, and/or decreased byproducts.

### BRIEF DESCRIPTION OF THE DRAWING

FIG. 1 is an illustration of one approach for the simultaneous synthesis of piperazine and methylated piperazine.

### DESCRIPTION

The present invention meets this need by providing piperazine having improved solubility in water. This provides increased operational flexibility and improved performance at higher CO₂ recovery.

U.S. Application Ser. No. 63/737,268, entitled “CATALYTIC METHOD FOR SELECTIVE CYCLO-AMINATION OF ALKANOLAMINE AND DIOL TO PRODUCE ON-PURPOSE CYCLIC ETHYLENEAMINES OF PIPERAZINE AND DERIVATIVES” filed on Dec. 20, 2024 and incorporated herein in its entirety, describes catalytic processes for the on-purpose production of piperazine. The processes are based on the use of a 10-member ring zeolite, preferably with MFI topology, with tuned morphology and acidity to promote intermolecular or intramolecular cyclization of alkanolamine, ethyleneamine, or diol, to cyclic piperazine. The processes are cost effective and have higher yield of piperazine than current processes. The processes described in U.S. Application Ser. No. 63/737,268 entitled “CATALYTIC METHOD FOR SELECTIVE CYCLO-AMINATION OF ALKANOLAMINE AND DIOL TO PRODUCE ON-PURPOSE CYCLIC ETHYLENEAMINES OF PIPERAZINE AND DERIVATIVES”, filed on Dec. 20, 2024 are the basis for obtaining a co-mixture of piperazine and methyl-substituted piperazine.

Alkylated piperazine (in the form of methylated C-substituted piperazine, e.g., 2-methyl piperazine or 2,5-dimethyl piperazine) has been identified as a piperazine additive which improved the aqueous solubility limitation of piperazine (see e.g., U.S. Pat. No. 8,816,078 B2). Mixtures of piperazine and 2-methyl piperazine can substantially prevent piperazine precipitation because solid precipitation typically occurs as a highly organized crystal, and minor changes in molecular structure will prevent the coprecipitation of closely related molecules.

The present application describes catalytic processes to manufacture piperazine and 2-methyl piperazine simultaneously through co-feeding an ethanolamine, such as monoethanolamine (MEA), or diethanolamine (DEA)), a diol, such as ethylene glycol, or an ethyleneamine, such as ethylenediamine (EDA) with a C₃ analog having a different degree of amination over a selective catalyst comprising a 10 MR MFI topology with controlled acidity and morphology. The acidity of the catalyst is controlled by minimizing acid sites on the external surface and maintaining a higher ratio of strong acid sites to weaker ones. This can be obtained by proper calcination, the addition of phosphorus, metal incorporation (e.g., by aqueous impregnation), and/or surface passivation using SiO₂. With respect to morphology, the catalyst has dispersed crystals with a smaller particle size, e.g., 1-2 microns.

Examples of C₃ analogs include, but are not limited to, monopropylene glycol (MPG), monoisopropylamine (MIPA), or propylenediamine (PDA).

One aspect of the invention is a process for the production of a co-mixture of piperazine and methyl-substituted piperazine. In one embodiment, the process comprises co-feeding a linear alkanolamine, or a diol, or an ethyleneamine with a analog of the alkanolamine, or the diol, or the ethyleneamine in a reaction zone comprising a reactor in the presence of anhydrous ammonia and a catalyst comprising a 10-member ring zeolite to produce a reaction mixture comprising the piperazine and the methyl-substituted piperazine.

In some embodiments, the alkanolamine comprises diethanolamine, or monoethanolamine, or triethanolamine, or combinations thereof.

In some embodiments, the analog of the alkanolamine comprises monoisopropanolamine.

In some embodiments, the diol comprises ethylene glycol.

In some embodiments, the analog of the diol comprises monopropylene glycol.

In some embodiments, the ethyleneamine comprises ethylenediamine.

In some embodiments, the analog of the ethylenediamine comprises propylenediamine.

In some embodiments, a ratio of the linear alkanolamine, or the diol, or the ethylenediamine to the analog of the alkanolamine, or the diol, or the ethylenediamine in the feed is in a range of 40:60 to 85:15.

In some embodiments, the 10-member ring zeolite comprises an MFI-type zeolite.

In some embodiments, the 10-member ring zeolite is modified with phosphorous.

In some embodiments, the phosphorous comprises phosphoric acid, phosphorous oxide, or ammonium dihydrogen phosphate.

In some embodiments, the catalyst has an acidity in a range of 0.75 mmol/g to 1.2 mmol/g.

In some embodiments, the catalyst has a mole ratio of Si to Al in a range of 10 to 140.

In some embodiments, the reaction conditions comprise a temperature in a range of 280° C. to 350° C., or a pressure in a range of 1700-2500 psig, or both.

In some embodiments, a mole ratio of the anhydrous ammonia to the alkanolamine or the diol or the ethylenediamine is in a range of 10 to 100.

In some embodiments, the reaction mixture further comprises a piperazine derivative and further comprising: separating the reaction mixture into a piperazine product stream comprising the piperazine and the methyl-substituted piperazine, and a byproduct stream comprising the piperazine derivative; and recycling the byproduct stream to the reaction zone.

In some embodiments, the piperazine derivative comprises at least one of an alkylated piperazine having an alkyl chain with 2 or more carbon atoms, triethylene diamine, aminoethylpiperazine, hydroxyethylpiperazine.

In some embodiments, the reaction mixture comprises a weight ratio of the piperazine to the methyl-substituted piperazine in a range of 10:90 to 90:10.

In some embodiments, the methyl-substituted piperazine comprises N-methyl piperazine, N,N′-dimethylpiperazine, or combinations thereof. All isomers of dimethylpiperazine can be formed, e.g., cis, trans, and racemic mixtures.

Another aspect of the invention is a process for the production of a co-mixture of piperazine and methyl-substituted piperazine. In one embodiment, the process comprises co-feeding a linear alkanolamine, or a diol, or an ethyleneamine with an analog of the alkanolamine, or the diol, or the ethyleneamine in a reaction zone comprising a reactor in the presence of anhydrous ammonia and a catalyst comprising a 10-member ring zeolite to produce a reaction mixture comprising the piperazine, the methyl-substituted piperazine, and a piperazine derivative, wherein the reaction mixture comprises a weight ratio of the piperazine to the methyl-substituted piperazine in a range of 10:90 to 90:10. The reaction mixture is separated into a piperazine product stream comprising the piperazine and the methyl-substituted piperazine, and a byproduct stream comprising the piperazine derivatives. The byproduct stream is recycled to the reaction zone.

In some embodiments, the alkanolamine comprises diethanolamine, or monoethanolamine, or triethanolamine, or combinations thereof, and the analog of the alkanolamine comprises monoisopropanolamine; or the diol comprises ethylene glycol and the analog of the diol comprises monopropylene glycol; or the ethyleneamine comprises ethylenediamine and the analog of the ethylenediamine comprises propylenediamine; or the piperazine derivative comprises at least one of an alkylated piperazine having an alkyl chain with 2 or more carbon atoms, triethylene diamine, aminoethylpiperazine, hydroxyethylpiperazine; or combinations thereof.

In some embodiments, the 10-member ring zeolite comprises an MFI-type zeolite; or 10-member ring zeolite is modified with phosphorous or by silylation; or the catalyst has an acidity in a range of 0.75 mmol/g to 1.2 mmol/g; or the catalyst has a mole ratio of Si to Al in a range of 10 to 140; or combinations thereof.

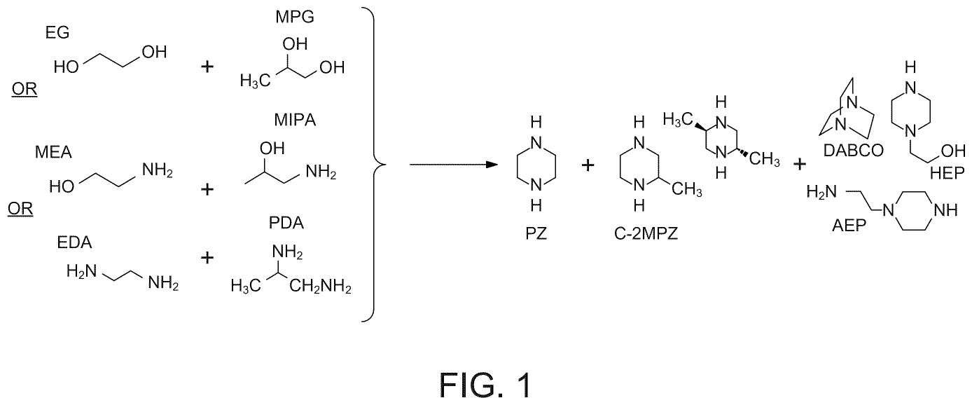

FIG. 1 illustrates the technical approach to synthesize a co-mixture of piperazine (PZ) and 2-methylpiperazine (2-MPZ) which utilizes co-feeding a C₃ analog, such as monopropylene glycol (MPG), monoisopropanolamine (MIPA), or propylene diamine (PDA) with a linear alkanolamine (MEA, DEA), diol (ethylene glycol (EG)), or ethylenediamine (EDA). FIG. 1 also illustrates some of the piperazine derivatives that can be formed in the reaction, such as triethylenediamine (TEDA (DABCO)), aminoethylpiperazine (AEP), and hydroxyethylpiperazine (HEP).

One potential catalyst is an MFI zeolite with low Si/Al ratio (e.g., less than or equal to 11), as described in U.S. Application Ser. No. 63/737,268, entitled “CATALYTIC METHOD FOR SELECTIVE CYCLO-AMINATION OF ALKANOLAMINE AND DIOL TO PRODUCE ON-PURPOSE CYCLIC ETHYLENEAMINES OF PIPERAZINE AND DERIVATIVES”. The ratio of feed/C₃ analog helps control the total (piperazine+2-methyl piperazine) and the ratio. The total amount of piperazine and 2-methyl piperazine is typically less than or equal to 70 wt % (selectivity). The ratio of piperazine to 2-methyl piperazine is in the range of 10:90 to 90:10. It is worth noting that methyl-substituted piperazine (2-methyl piperazine) cannot be formed by the simple amination/cyclization reactions of EDA, MEA, DEA, or EG. The C₃ analog is needed to produce methyl substituted piperazine.

### EXAMPLES

The following examples illustrate aspects of the invention.

### Example 1

Selective cyclo-amination of mixtures containing diol (ethylene glycol, EG), alkanolamine (monoethanolamine (MEA), diethanolamine (DEA) or ethyleneamine (ethylenediamine, EDA) with the corresponding C₃ analog-monopropylene glycol (MPG), monoisopropylamine (MIPA), or propylenediamine (PDA), was conducted in a high pressure stirred SS316 batch reactor. The ratio of feed to C₃ analog was in the range of 40:60 to 85:15. The catalyst, which was added in powdered form, was activated ex.situ at 275° C. for 10 hours under nitrogen flow. Examples of catalysts include MFI zeolite of variable SiO₂/Al₂O₃ ratio (23-280) (MFI23, 23 represents SiO₂/Al₂O₃); small (50-100 nm) and large (2 microns) MFI crystals; FAU zeolite (HY5, USY30), metal-containing MFI. A certain amount of feed mixture and liquified anhydrous ammonia were charged into the vessel, with a molar ration of NH₃ to feed of 10-100. The mixture was heated (280-350° C.) under stirring for 30 min to 10 hours on stream under autogenic pressure (1700-2500 psig). Liquid products were analyzed offline using gas chromatography equipped with flame ionization detector (FID) and CP-Volamine 60 m×0.32 mm i.d×5 μm.

Gas chromatography analysis of the products from the (EG+C₃ analog) animation is shown in Table 1. Selectivity to (PZ+2-MPZ) is dependent on zeolite topology, Si/Al ratio, acidity, reaction parameters, and feed/C₃ ratio. Ethylene glycol is a low reactive feed for amination to piperazine and co-mixture of piperazine and 2-methylpiperazine, compared to monoethanolamine, diethanolamine, and ethylenediamine (Example 2). Propylenediamine (PDA) demonstrates the best C₃ analog to obtain high selectivity to a desirable mixture of (PZ+2-MPZ) as high as 65 wt. %, followed by MIPA and MPG (53 wt. %, 37.4 wt. %) at comparable conversion level. It was shown that the selectivity of an inherently low reactive MPG based mixture to (PZ+2-MPZ) can be increased to match that with PDA by altering the reaction duration and increasing the zeolite acidity.

TABLE 1
Synthesis of co-mixture of PZ + 2-MPZ through selective
cyclo-amination of mixture containing linear ethylene glycol (EG) with
C₃ analog (300° C., 10 HOS, 145-152 barg (autogenic NH₃))

| | | | | Product Selectivity (%) | | | | |
|---|---|---|---|---|---|---|---|---|
| | C3 | Feed/ | X_{feed} | | | PZ + 2- | | PZ/ |
| Catalyst | analog | C3 | (%) | PZ | 2-MPZ | MPZ | Amination ^{a} | 2-MPZ |
| MFI23 (18) | MPG | 50:50 | 36.5 | 19.6 | 17.8 | 37.4 | 12.9 | 52/48 |
| MFI23 (18) | MPG | 50:50 | 34.4 | 33.9 | 26.7 | 60.6 | 15.7 | 56/44 |
| (20 HOS) | | | | | | | | |
| MFI23 (18) | MPG | 50:50 | 11.4 | 4.3 | — | 4.30 | — | 100/— |
| (4 HOS) | | | | | | | | |
| MFI23 (16) | MIPA | 50:50 | 37.9 | 11.4 | 41.6 | 53.0 | 30.3 | 22/78 |
| 2% Ga/MFI40 | MIPA | 50:50 | 23.3 | 5.92 | — | 5.92 | 77.3 | 100/— |
| (18) | | | | | | | | |
| MFI23 (18) | PDA | 25:75 | 53.3 | 20.1 | 34.1 | 54.2 | — | 37/63 |
| MFI23 (19) | PDA | 50:50 | 41.1 | 16.8 | 46.6 | 63.4 | — | 27/73 |
| MFI23 (17) | PDA | 65:35 | 37.5 | 18.9 | 48.3 | 67.2 | — | 28/72 |
| MFI23 (17) | PDA | 85:15 | 35.1 | 24.1 | 43.7 | 67.8 | — | 36/64 |
| MFI23 (17) | PDA | 85:15 | 18.1 | 22.8 | 48.3 | 71.1 | — | 32/68 |
| (4 HOS) | | | | | | | | |
| MFI23 (16) | PDA | 90:10 | 20.9 | 20.9 | 32.9 | 53.8 | — | 39/61 |
| HY5 (17) | PDA | 50:50 | 60.9 | 20.5 | 53.0 | 73.5 | — | 28/72 |
| (4 HOS) | | | | | | | | |
| USY30 (18) | PDA | 50:50 | 51.7 | 21.3 | 25.9 | 47.2 | — | 45/55 |
| (4 HOS) | | | | | | | | |

Number in parentheses represents NH₃/feed molar ratio. X_{feed} - conversion.
^{a} Amination products (MIPA + PDA)

### Example 2

Mixtures containing monoethanolamine (MEA) or ethylenediamine (EDA) and C₃ analog were cyclo-aminated according to the procedures used in Example 1. Lower reaction duration is considered due to the high reactivity of alkanolamine and ethylenediamine compared to ethylene glycol. Similar to the EG based mixture, animation with PDA showed the highest (PZ+2-MPZ) selectivity (Table 2), whereas (un-aminated, partially aminated) C₃ analogs can be tuned to obtain high (PZ+2-MPZ) selectivity. The addition of anhydrous ammonia to inherently fully aminated (EDA+PDA) based mixtures enhanced conversion and selectivity to the desirable product with a 20-30% increase in selectivity to (PZ+2-MPZ). Amination products (i.e., MIPA, PDA) are not detected, primarily due to the high feed reactivity.

TABLE 2
Selective cyclo-amination of mixture containing linear monoethanolamine (MEA) or ethylenediamine
(EDA) with C₃ analog (300° C., 4 HOS, 145-152 barg (autogenic NH₃))

| | | | | | Product Selectivity (%) | | | | |
|---|---|---|---|---|---|---|---|---|---|
| | | C3 | Feed/ | X_{feed} | | | PZ + 2- | | PZ/ |
| Catalyst | Feed | analog | C3 | (%) | PZ | 2-MPZ | MPZ | Amination^{a} | 2-MPZ |
| MFI23 (17) | MEA | MIPA | 50:50 | 67.5 | 21.6 | 40.9 | 62.5 | — | 35/65 |
| MFI30N^{b} (16) | MEA | MIPA | 50:50 | 70.4 | 18.7 | 38.2 | 56.9 | — | 33/67 |
| MFI30L^{c} (14) | MEA | MIPA | 50:50 | 47.1 | 24.5 | 38.1 | 62.6 | — | 39/61 |
| 2% Ga/MFI40 (17) | MEA | MIPA | 50:50 | 27.7 | 21.0 | 23.5 | 44.5 | — | 47/53 |
| HY5 (17) | MEA | MIPA | 50:50 | 73.2 | 20.9 | 47.0 | 67.9 | — | 31/69 |
| MFI23 (16) | MEA | MIPA | 20:80 | 47.0 | 17.4 | 50.5 | 67.9 | — | 26/74 |
| 2% Ga/MFI40 (11) | MEA | MIPA | 80:20 | 26.7 | 19.9 | — | 19.9 | — | 100/— |
| MFI23 (30) | MEA | PDA | 50:50 | 87.2 | 14.8 | 51.3 | 66.1 | — | 22/78 |
| MFI23 (no NH₃) | EDA | MIPA | 50:50 | 31.2 | 26.5 | 24.5 | 51.0 | | 52/48 |
| MFI23 (15) | EDA | MIPA | 50:50 | 67.8 | 19.0 | 46.8 | 65.8 | — | 29/71 |
| HY5 (18) | EDA | MIPA | 50:50 | 48.4 | 21.8 | 54.0 | 75.8 | — | 29/71 |
| MFI23 (no NH₃) | EDA | PDA | 50:50 | 71.5 | 16.8 | 49.6 | 66.4 | | 25/75 |
| MFI23 (17) | EDA | PDA | 50:50 | 92.1 | 24.5 | 56.3 | 80.8 | | 30/70 |
| 2% Ga/MFI40 | EDA | PDA | 50:50 | 20.1 | 13.9 | 26.7 | 40.6 | — | 34/66 |
| (no NH₃) | | | | | | | | | |
| 2% Ga/MFI40 (no | EDA | PDA | 50:50 | 35.7 | 13.4 | 27.2 | 40.6 | — | 33/67 |
| NH₃) (10 HOS) | | | | | | | | | |
| 2% Ga/MFI40 (12) | EDA | PDA | 50:50 | 43.1 | 20.8 | 31.3 | 52.1 | — | 40/60 |

Number in parentheses represents NH₃/feed molar ratio. X_{feed} - conversion.
^{a}Amination products (MIPA + PDA).
^{b}nanocrystals;
^{c}large crystals

### Example 3

Cyclo-amination of mixtures containing diethanolamine (DEA) and C₃ analog of ethylene glycol, monoethanolamine or ethylenediamine, similar to procedures used in Example 1. C₄ analog includes Aminoethylethanolamine (AEEA). As shown in Table 3, the selectivity to (PZ+2-MPZ) for PDA based mixtures remains the highest compared to MPG and MIPA. DEA based mixtures show lower performance compared to EDA and MEA, but higher than EG mixtures, illustrating the importance of feed reactivity and composition.

TABLE 3
Selective cyclo-amination of mixture containing diethanolamine
(DEA) and C3 analog of diol, alkanolamine, or ethylenediamine (300°
C., 4 HOS, 145-152 barg (autogenic NH₃))

| | | | | | Product Selectivity (%) | | | | |
|---|---|---|---|---|---|---|---|---|---|
| | | C3 | Feed/ | X_{feed} | | 2- | PZ + 2- | | PZ/ |
| Catalyst | Feed | analog ^{a} | C3 | (%) | PZ | MPZ | MPZ | Amination ^{b} | 2MPZ |
| MFI23 (48) | DEA | MPG | 50:50 | 36.1 | 42.4 | — | 42.4 | 6.03 | — |
| MFI23 (40) | DEA | MPG | 40:60 | 31.6 | 33.3 | 17.3 | 50.6 | 14.4 | 66/34 |
| MFI23 (80) | DEA | MPG | 40:60 | 45.4 | 17.3 | 18.3 | 35.6 | 24.3 | 49/51 |
| MFI23 (40) | DEA | MIPA | 40:60 | 93.6 | 13.1 | 38.4 | 51.5 | 24.9 | 25/75 |
| MFI80 (40) | DEA | MIPA | 40:60 | 76.4 | 19.1 | 27.1 | 46.2 | 26.3 | 41/59 |
| 2% FeMFI30 | DEA | MIPA | 40:60 | 71.5 | 17.4 | 26.3 | 43.7 | 33.1 | 40/60 |
| (40) | | | | | | | | | |
| MFI23 (45) | DEA | PDA | 40:60 | 95.1 | 22.5 | 35.1 | 57.6 | — | 39/61 |

Number in parentheses represents NH₃/feed molar ratio. X_{feed} - conversion.
^{a} analog of diol, ethanolamine, and ethylenediamine.
^{b} Amination products (MIPA + PDA)

### Example 4

### Comparative Example

(U.S. Application Ser. No. 63/737,268)—Amination of diol, alkanolamine, ethylenediamine, or diethanolamine without C₃ analog was carried out according to procedures used in Example 1. 2-MPZ was not detected (only piperazine and piperazine derivatives), since the addition of methyl group formed during the reaction is preferentially favorable at N position instead of C position.

### Specific Embodiments

While the following is described in conjunction with specific embodiments, it will be understood that this description is intended to illustrate and not limit the scope of the preceding description and the appended claims.

A first embodiment of the invention is a process for the production of a co-mixture of piperazine and methyl-substituted piperazine comprising co-feeding a linear alkanolamine, or a diol, or an ethyleneamine with an analog of the alkanolamine, or the diol, or the ethyleneamine in a reaction zone comprising a reactor in the presence of anhydrous ammonia and a catalyst comprising a 10-member ring zeolite to produce a reaction mixture comprising the piperazine and the methyl-substituted piperazine. An embodiment of the invention is one, any or all of prior embodiments in this paragraph up through the first embodiment in this paragraph wherein the alkanolamine comprises diethanolamine, or monoethanolamine, or triethanolamine, or combinations thereof. An embodiment of the invention is one, any or all of prior embodiments in this paragraph up through the first embodiment in this paragraph wherein the analog of the alkanolamine comprises monoisopropanolamine. An embodiment of the invention is one, any or all of prior embodiments in this paragraph up through the first embodiment in this paragraph wherein the diol comprises ethylene glycol. An embodiment of the invention is one, any or all of prior embodiments in this paragraph up through the first embodiment in this paragraph wherein the analog of the diol comprises monopropylene glycol. An embodiment of the invention is one, any or all of prior embodiments in this paragraph up through the first embodiment in this paragraph wherein the analog of the ethyleneamine comprises ethylenediamine. An embodiment of the invention is one, any or all of prior embodiments in this paragraph up through the first embodiment in this paragraph wherein the analog of the ethylenediamine comprises propylenediamine. An embodiment of the invention is one, any or all of prior embodiments in this paragraph up through the first embodiment in this paragraph wherein a ratio of the linear alkanolamine, or the diol, or the ethylenediamine to the analog of the alkanolamine, or the diol, or the ethylenediamine in the feed is in a range of 40:60 to 85:15. An embodiment of the invention is one, any or all of prior embodiments in this paragraph up through the first embodiment in this paragraph wherein the 10-member ring zeolite comprises an MFI-type zeolite. An embodiment of the invention is one, any or all of prior embodiments in this paragraph up through the first embodiment in this paragraph wherein the 10-member ring zeolite comprises an MFI-type zeolite, with mole ratio of Si to Al in a range of 10 to 140. An embodiment of the invention is one, any or all of prior embodiments in this paragraph up through the first embodiment in this paragraph wherein the 10-member ring zeolite is modified with phosphorous, metal incorporation, or by silylation. An embodiment of the invention is one, any or all of prior embodiments in this paragraph up through the first embodiment in this paragraph wherein the phosphorous comprises phosphoric acid, phosphorous oxide, or ammonium dihydrogen phosphate. An embodiment of the invention is one, any or all of prior embodiments in this paragraph up through the first embodiment in this paragraph wherein the catalyst has an acidity in a range of 0.75 mmol/g to 1.2 mmol/g. An embodiment of the invention is one, any or all of prior embodiments in this paragraph up through the first embodiment in this paragraph wherein the catalyst has a mole ratio of Si to Al in a range of 10 to 140. An embodiment of the invention is one, any or all of prior embodiments in this paragraph up through the first embodiment in this paragraph wherein the reaction conditions comprise a temperature in a range of 280° C. to 350° C., or a pressure in a range of 1700-2500 psig, or both. An embodiment of the invention is one, any or all of prior embodiments in this paragraph up through the first embodiment in this paragraph wherein a mole ratio of the anhydrous ammonia to the alkanolamine or the diol or the ethyleneamine is in a range of 10 to 100. An embodiment of the invention is one, any or all of prior embodiments in this paragraph up through the first embodiment in this paragraph wherein the reaction mixture further comprises a piperazine derivative and further comprising separating the reaction mixture into a piperazine product stream comprising the piperazine and the methyl-substituted piperazine, and a byproduct stream comprising the piperazine derivative; and recycling the byproduct stream to the reaction zone. An embodiment of the invention is one, any or all of prior embodiments in this paragraph up through the first embodiment in this paragraph wherein the piperazine derivative comprises at least one of an alkylated piperazine having an alkyl chain with 2 or more carbon atoms, triethylene diamine, aminoethylpiperazine, hydroxyethylpiperazine. An embodiment of the invention is one, any or all of prior embodiments in this paragraph up through the first embodiment in this paragraph wherein the reaction mixture comprises a weight ratio of the piperazine to the methyl-substituted piperazine in a range of 10:90 to 90:10. An embodiment of the invention is one, any or all of prior embodiments in this paragraph up through the first embodiment in this paragraph wherein the methyl-substituted piperazine comprises 2-methyl piperazine, 2,5-dimethylpiperazine, or combinations thereof.

A second embodiment of the invention is a process for the production of a co-mixture of piperazine and methyl-substituted piperazine comprising co-feeding a linear alkanolamine, or a diol, or an ethyleneamine with an analog of the alkanolamine, or the diol, or the ethyleneamine in a reaction zone comprising a reactor in the presence of anhydrous ammonia and a catalyst comprising a 10-member ring zeolite to produce a reaction mixture comprising the piperazine, the methyl-substituted piperazine, and a piperazine derivative, wherein the reaction mixture comprises a weight ratio of the piperazine to the methyl-substituted piperazine in a range of 1090 to 9010; separating the reaction mixture into a piperazine product stream comprising the piperazine and the methyl-substituted piperazine, and a byproduct stream comprising the piperazine derivatives; and recycling the byproduct stream to the reaction zone. An embodiment of the invention is one, any or all of prior embodiments in this paragraph up through the second embodiment in this paragraph the alkanolamine comprises diethanolamine, or monoethanolamine, or triethanolamine, or combinations thereof, and the analog of the alkanolamine comprises monoisopropanolamine; or the diol comprises ethylene glycol and the analog of the diol comprises monopropylene glycol; or the ethyleneamine comprises ethylenediamine and the analog of the ethylenediamine comprises propylenediamine; or the piperazine derivative comprises at least one of an alkylated piperazine having an alkyl chain with 2 or more carbon atoms, triethylene diamine, aminoethylpiperazine, hydroxyethylpiperazine; or combinations thereof. An embodiment of the invention is one, any or all of prior embodiments in this paragraph up through the second embodiment in this paragraph wherein the 10-member ring zeolite comprises an MFI-type zeolite; or wherein the 10-member ring zeolite is modified with phosphorous, metal incorporation, or by silylation; or wherein the catalyst has an acidity in a range of 0.75 mmol/g to 1.2 mmol/g; or wherein the catalyst has a mole ratio of Si to Al in a range of 10 to 140; or combinations thereof.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process for the production of a co-mixture of piperazine and methyl-substituted piperazine comprising:
co-feeding a linear alkanolamine, or a diol, or an ethyleneamine with an analog of the alkanolamine, or the diol, or the ethyleneamine in a reaction zone comprising a reactor in the presence of anhydrous ammonia and a catalyst comprising a 10-member ring zeolite to produce a reaction mixture comprising the piperazine and the methyl-substituted piperazine.

2. The process of claim 1 wherein the alkanolamine comprises diethanolamine, or monoethanolamine, or triethanolamine, or combinations thereof.

3. The process of claim 2 wherein the analog of the alkanolamine comprises monoisopropanolamine.

4. The process of claim 1 wherein the diol comprises ethylene glycol.

5. The process of claim 1 wherein the analog of the diol comprises monopropylene glycol.

6. The process of claim 1 wherein the analog of the ethyleneamine comprises ethylenediamine.

7. The process of claim 6 wherein the analog of the ethylenediamine comprises propylenediamine.

8. The process of claim 1 wherein a ratio of the linear alkanolamine, or the diol, or the ethylenediamine to the analog of the alkanolamine, or the diol, or the ethylenediamine in the feed is in a range of 40:60 to 85:15.

9. The process of claim 1 wherein the 10-member ring zeolite comprises an MFI-type zeolite, with mole ratio of Si to Al in a range of 10 to 140.

10. The process of claim 1 wherein the 10-member ring zeolite is modified with phosphorous, metal incorporation, or by silylation.

11. The process of claim 1 wherein the phosphorous comprises phosphoric acid, phosphorous oxide, or ammonium dihydrogen phosphate.

12. The process of claim 1 wherein the catalyst has an acidity in a range of 0.75 mmol/g to 1.2 mmol/g.

13. The process of claim 1 wherein the reaction conditions comprise a temperature in a range of 280° C. to 350° C., or a pressure in a range of 1700-2500 psig, or both.

14. The process of claim 1 wherein a mole ratio of the anhydrous ammonia to the alkanolamine or the diol or the ethyleneamine is in a range of 10 to 100.

15. The process of claim 1 wherein the reaction mixture further comprises a piperazine derivative and further comprising:
separating the reaction mixture into a piperazine product stream comprising the piperazine and the methyl-substituted piperazine, and a byproduct stream comprising the piperazine derivative; and
recycling the byproduct stream to the reaction zone.

16. The process of claim 15 wherein the piperazine derivative comprises at least one of an alkylated piperazine having an alkyl chain with 2 or more carbon atoms, triethylene diamine, aminoethylpiperazine, hydroxyethylpiperazine.

17. The process of claim 1 wherein the reaction mixture comprises a weight ratio of the piperazine to the methyl-substituted piperazine in a range of 10:90 to 90:10; or wherein the methyl-substituted piperazine comprises 2-methyl piperazine, 2,5-dimethylpiperazine, or combinations thereof; or both.

18. A process for the production of a co-mixture of piperazine and methyl-substituted piperazine comprising:
co-feeding a linear alkanolamine, or a diol, or an ethyleneamine with an analog of the alkanolamine, or the diol, or the ethyleneamine in a reaction zone comprising a reactor in the presence of anhydrous ammonia and a catalyst comprising a 10-member ring zeolite to produce a reaction mixture comprising the piperazine, the methyl-substituted piperazine, and a piperazine derivative, wherein the reaction mixture comprises a weight ratio of the piperazine to the methyl-substituted piperazine in a range of 10:90 to 90:10;
separating the reaction mixture into a piperazine product stream comprising the piperazine and the methyl-substituted piperazine, and a byproduct stream comprising the piperazine derivatives; and
recycling the byproduct stream to the reaction zone.

19. The process of claim 18:
the alkanolamine comprises diethanolamine, or monoethanolamine, or triethanolamine, or combinations thereof, and the analog of the alkanolamine comprises monoisopropanolamine; or
the diol comprises ethylene glycol and the analog of the diol comprises monopropylene glycol; or
the ethyleneamine comprises ethylenediamine and the analog of the ethylenediamine comprises propylenediamine; or
the piperazine derivative comprises at least one of an alkylated piperazine having an alkyl chain with 2 or more carbon atoms, triethylene diamine, aminoethylpiperazine, hydroxyethylpiperazine; or
combinations thereof.

20. The process of claim 18:
wherein the 10-member ring zeolite comprises an MFI-type zeolite; or
wherein the 10-member ring zeolite is modified with phosphorous, metal incorporation, or by silylation; or
wherein the catalyst has an acidity in a range of 0.75 mmol/g to 1.2 mmol/g; or
wherein the catalyst has a mole ratio of Si to Al in a range of 10 to 140; or
combinations thereof.
